(19) United States
(12) Patent Application Publication
(10) Pub. No.: US 2021/0061757 A1
(43) Pub. Date: Mar. 4, 2021

(54) PROCESS FOR THE SYNTHESIS OF OPTICALLY ACTIVE BETA-AMINO ALCOHOLS

(71) Applicant: OLON S.p.A., Rodano (IT)

(72) Inventors: Filippo NISIC, Rodano (MI) (IT); Faris GARIS, Rodano (MI) (IT); Corrado COLLI, Rodano (MI) (IT); Giorgio BERTOLINI, Rodano (MI) (IT); Mara SADA, Rodano (MI) (IT); Stefania BERTUOLO, Milano (MI) (IT); Silvano RONZONI, Milano (MI) (IT); Romano DI FABIO, Milano (MI) (IT); Stefano MAIORANA, Milano (MI) (IT)

(21) Appl. No.: 17/046,096

(22) PCT Filed: Apr. 11, 2019
(86) PCT No.: PCT/IB2019/052986

§ 371 Date: Oct. 8, 2020

(30) Foreign Application Priority Data
Apr. 13, 2018 (IT) 102018000004492

(51) Int. Cl. C07C 269/06; C07C 213/02

(52) U.S. Cl. CPC C07C 269/06; C07C 215/60; C07C 213/02

(57) ABSTRACT

Subject-matter of the present invention is a process for the preparation of optically active phenyl-beta-amino alcohols by means of a specific reduction of the corresponding phenyl-beta-amino ketones. Further subject-matter of the invention are said novel synthesis intermediates and their use for the preparation of active pharmaceutical ingredients.

### ABSTRACT OF THE INVENTION

Object of the present invention is a process for the preparation of optically active phenyl-beta-amino alcohols by means of a specific reduction of the corresponding phenyl-beta-amino ketones. Further subject-matter of the invention are said novel synthesis intermediates and their use for the preparation of active pharmaceutical ingredients.

### TECHNICAL FIELD

Amino alcohols, in particular the chiral phenyl-beta-amino alcohols, are very important synthons for the synthesis of active pharmaceutical ingredients; their basic structure is for example present in the epinephrine and norepinephrine hormones (also named adrenaline and nor-adrenaline), as well as in some drugs used for the treatment of asthma or chronic bronchitis (COPD) such as isoproterenol.

Optically active beta-amino alcohols are also of industrial interest as they can be used as chiral ligands or auxiliaries in different types of asymmetric syntheses. Due to the relevance of such molecules, several synthesis methods have been developed over the years.

Initially the most used synthesis route, as it is promising in terms of optical purity, was the chiral resolution by optically active chemical compounds of the racemic amino alcohol but unfortunately such a synthesis route was not convenient in terms of yield.

Recently different enantioselective synthesis methods have been developed, that are more effective than the resolution, in terms of yield.

The hydrogenation often involves the use of high pressures, expensive metal catalysts and often yields to impurities due to an excessive reduction (“overreduction”) or to side reactions on other parts of the molecule.

By way of example, with reference to the known syntheses of epinephrine (also named adrenaline), are known:
The resolution from the corresponding racemate by salification but this technology requires however a big waste of product and very low yields.
A chiral synthesis by means of a hydrogenation with a chiral catalyst based on ferrocene, as described in Tetrahedron Letters 5(1979), 425-428; unfortunately this technique, beside involving very high hydrogenation times and pressures, 2-4 days at 50 atm (about 50 bar), with resulting safety risks, is also economically poorly profitable. Indeed, apart from the very long duration of the reaction, with the resulting occupancy of the industrial equipment, it has to be highlighted that industrial equipment for the hydrogenation capable of reaching 50 bar are not of common use. In general, the normal reactors used in the chemical industry cannot go beyond 5-7 bar. In addition, the hydrogenators have often some limitations to 15-20 bar and some others to about 30 bar, but only very few can reach 50 bar and they often have capacities more similar to a pilot unit than to an industrial plant. The synthesis proposed in the above mentioned document is then barely accessible and usable to most of the chemical industries. Moreover, the same document at page 427 states that the proposed hydrogenation method is an alternative to the conventionally used chiral reduction method with hydrides and the use of borane is absolutely not considered for the reduction of phenyl-beta-amino ketones.
A chiral synthesis by means of a hydrogenation with a chiral catalyst based on rhodium and phosphines (as described in the Patent WO01/12583 and in its corresponding U.S. Pat. No. 6,218,575); this synthetic route, even though partially reducing the safety problems and the costs of the reduction with respect to the synthesis with ferrocene, requires in any case the use of hydrogen at high pressure. This involves therefore the use of special reactors capable of withstanding reactions under hydrogen pressure, therefore such reaction cannot be carried out on the most common reactors in the industrial chemical plants, which usually withstand pressures not higher than 6-7 bar.

There is therefore the need to provide a new synthesis route for the preparation of phenyl-beta-amino alcohols, such as epinephrine and analogue compounds, which solves the drawbacks of the prior art as those mentioned above.

### OBJECTS OF THE INVENTION

It is an object of the invention to provide a process suitable for the preparation of optically active phenyl-beta-amino alcohols, with good yields and high enantiomeric excesses, easily feasible also on an industrial scale.

It is another object of the invention to provide a process suitable for the preparation of optically active phenyl-beta-amino alcohols, which overcomes the drawbacks of the prior art, such as those reported above.

It is a further object of the invention to provide novel intermediates useful in particular, but not limited to, for the preparation of epinephrine and salts thereof.

### DESCRIPTION OF THE INVENTION

It has been found, surprisingly, that a specific reducing agent is capable of providing the reduction of phenyl-beta-amino ketones to optically active phenyl-beta-amino alcohols, in the desired isomeric form, with very high yields and enantiomeric excesses and without the need of working under industrially difficult or dangerous conditions.

Thus, according to one of its aspects, subject-matter of the invention is a process for the preparation of an optically active compound of formula (I)

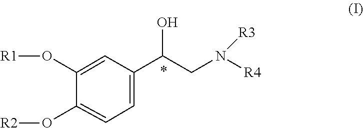

or a salt thereof, wherein
the asterisk means that the chiral carbon is in the optically active form (R) or (S);
R₁ e R₂ are, each independently, selected from hydrogen and a hydrox protecting group; or R₁ and R₂ together with the oxygen atoms to which they are bound, may form a protecting group in the form of a fused ring with benzene;
R₃ is selected from hydrogen and a protecting group of the amine function;
R₄ is selected from hydrogen and a C₁-C₄ alkyl; said process comprising

a. reducing the compound of Formula (II)

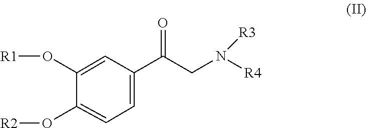

wherein R₁, R₂, R₃ e R₄ are as defined above and when R₃ is hydrogen, the amine group may be salified, said reduction being performed by a reducing complex made of phenylboronic acid or boranes in the presence of the Corey-Bakshi-Shibata (CBS) catalyst, in an organic solvent;

b. optionally, when R₁, R₂ and R₃ are protecting groups, removing said protecting groups to obtain the compound of formula (I) wherein R₁, R₂ and R₃ are hydrogen and R₄ is selected from hydrogen or a C₁-C₄ alkyl; and

c. optionally, converting the compound of formula (I) into a salt thereof;
steps (b) and (c) may be reversed.

The expression “chiral carbon is in the optically active (R) or (S) form” means herein that at least 80%, preferably at least 90-95%, more preferably 98-99.9% and up to 100%, of the compound of Formula (I) has said (R) or (S) configuration.

According to a preferred embodiment, the compound of formula (I) is in the (R) form.

The expressions “hydroxy protecting group” and “amine function protecting group” are well known to the person skilled in the art. Such protecting groups are for example described in T. W. Greene, John Wiley & Sons, Ltd, “Protective Groups in Organic Synthesis”, 5° edition, 2014.

According to a preferred embodiment, said hydroxy and amine function protecting groups are protecting groups which can be removed by hydrogenation or alkaline hydrolysis, advantageously by hydrogenation. In this last case, said protecting groups can be removed with hydrogen transfer techniques without the use of hydrogen under pressure, e.g. with formates or formic acid in the presence of a catalyst, such as for example palladium (Pd) or, alternatively, with hydrogen under pressure, in the presence of suitable catalysts or still with any other technique suitable to the purpose, as it is well known to the person skilled of the art.

Said protecting groups, each independently, are preferably selected from benzyl group and carbobenzyloxy group.

Preferably, said protecting groups can be removed by hydrogenation not at high pressure, such as for example with a maximum hydrogen pressure of 3.0±0.2 bar. More preferably, said removal by hydrogenation not at high pressure is carried out in the presence of a carboxylic acid which has at least one chiral center and is in an enantiomerically pure form, e.g. selected from D-tartaric acid, L-tartaric acid, D-benzoyltartaric acid, L-benzoyltartaric acid, D-camphor-10-sulfonic acid, L-camphor-10-sulfonic acid, D-mandelic acid, L-mandelic acid and the like, advantageously in the presence of tartaric acid in optically pure form.

In this embodiment the acid is used in equimolar amount or in slight excess relative to the compound to be deprotected, e.g. in an excess of 5-10%. The so-obtained salified deprotected product can be isolated, if desired or required, directly subjected to hydrolysis, according to methods well known in the art, to obtain the unsalified compound of formula (I).

Indeed it has been surprisingly observed that the use of such acids in the above mentioned reaction, preferably of tartaric acid in optically pure form, produces significant advantages in terms of better yield, product purity and enantiomeric purity.

According to a preferred embodiment, R₁ and R₂ are the same.

According to a preferred embodiment, R₁ and R₂ do not both represent hydrogen.

According to a more preferred embodiment, R₁ and R₂ are the same and each represents a benzyl group.

According to a preferred embodiment, R₃ represents a carbobenzyloxy group.

According to a preferred embodiment, R₁ and R₂ are the same and each represents a benzyl group and R₃ represents a carbobenzyloxy group.

According to a preferred embodiment, R₁, R₂ and R₃ are the same and each represents a carbobenzyloxy group.

According to a preferred embodiment, R₁, R₂ and R₃ are the same and each represents a carbobenzyloxy group and R₄ is a methyl group.

According to a preferred embodiment, R₃ is a protecting group which can be removed by hydrogenation, preferably a carbobenzyloxy group and R₄ is hydrogen.

The term “alkyl” means herein a saturated, linear or branched alkyl residue, having preferably 1 to 4 carbon atoms, advantageously from 1 to 4 carbon atoms, e.g. the methyl, ethyl, isopropyl, t-butyl group. Preferred alkyl groups are methyl, isopropyl and t-butyl.

According to a preferred embodiment, R₃ is a protecting group which can be removed by hydrogenation, preferably a carbobenzyloxy group and R₄ is a methyl group.

According to another preferred embodiment, R₃ and R₄ each represents a benzyl group, R₃ is hydrogen or a carbobenzyloxy and R₄ is a methyl group.

When the compound of formula (II) is in the form of a salt thereof, the counter-ion can be any anion derived from an organic or inorganic acid, such as for example formic acid, acetic acid, hydrochloric acid, hydrobromic acid, sulfuric acid and the like.

According to another preferred embodiment, R₃ is hydrogen and the compound of formula (II) is in salified form, advantageously in the form of hydrochloride or hydrobromide salt.

According to another preferred embodiment, R₁ and R₂ are each a benzyl group, R₃ is hydrogen, R₄ is a methyl and the compound of formula (I) is in salified form, advantageously in the hydrochloride form.

The CBS catalyst used in step (a) of the process of the invention is known in the art and is commercially available.

According to a preferred embodiment, the reaction of step (a) is carried out with CBS and borane (BH₃). Preferably, the borane is used in complexed form with dimethyl sulfide, e.g. in the form of a borane-dimethyl sulfide solution in a suitable solvent, advantageously in tetrahydrofuran. Such solution is known in the art and is also commercially available. The BH₃-CBS reducing complex can be formed in situ, as it will be described in the following Experimental Section.

The solvent used in step (a) may be any suitable organic solvent, preferably of aprotic type, such as for example an alkane, such as pentane, hexane cyclohexane; an aromatic hydrocarbon, such as for example benzene, toluene, xylene; dimethylformamide, dimethyl sulfoxide, dioxane, tetrahydrofuran and the like. Solvent mixtures can be obviously used. The solvent is preferably selected from toluene and tetrahydrofuran. A particularly preferred solvent is toluene.

The reaction of step (a) is advantageously carried out at low temperature, e.g. at a temperature from −5° C. to +5° C., preferably by first preparing in situ the complex in a suitable solvent, e.g. in toluene and then by adding slowly to the mixture the compound of formula (II). The amount of reducing complex used is advantageously stoichiometric or substoichiometric; for example 0.2-0-3 to 1.5 equivalents of reducing complex with respect to the compound of formula (II) can be used.

The compound of formula (I) obtained in step (a) can be isolated and purified, or used as such in the following possible step (b) and/or (c).

The removal of the R₁, R₂ and R₃ protecting groups can be carried out simultaneously or in two separate steps. When the protecting groups can be for example removed by hydrogenation, such as in the case of benzyl or carbobenzyloxy, they can be removed with a single reaction.

The reactions of steps (b) and (c) are known to the person skilled in the art; however details of the preferred conditions are provided in the following Experimental Section.

Some compounds of formula (I) and (II) are known in the art, while the compounds having the following formulas are novel:

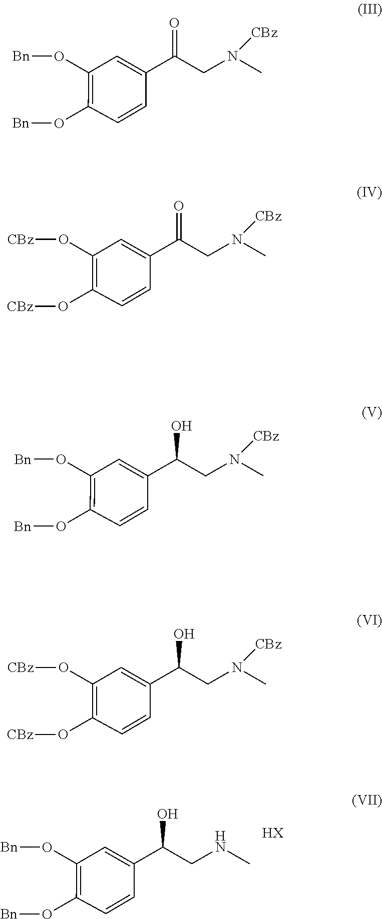

wherein X represents a halogen atom, advantageously bromine and chlorine, preferably chlorine, the compounds (V), (VI) and (VII) may be in the form of racemates, pure isomers or isomer mixtures, preferably in the form of (R) isomer.

Such compounds are a further subject-matter of the present invention as well as their use as synthesis intermediates, in particular but not only, in the preparation of the compounds of formula (I) wherein R₁, R₂ and R₃ are hydrogen, advantageously in the preparation of epinephrine (also named adrenaline).

Therefore the process of the invention allows optically active phenyl-beta-amino alcohols to be obtained, in the “R” form, such as for example the epinephrine (or adrenaline), the norepinephrine (or noradrenaline) and the isoproterenol.

The process of the invention to obtain the epinephrine is a preferred embodiment of the invention, more preferably the process of the invention wherein R₁ and R₂ are the same and each represents a benzyl group and R₃ represents a carbobenzyloxy group, and wherein said protecting groups are removed by hydrogenation at a not high pressure, e.g. with a maximum hydrogen pressure of 3.0±0.2 bar, and in presence of L-tartaric acid.

As it will be described in detail in the Experimental Section, the process of the invention provides the compounds of formula (I) with surprising yields and enantiomeric excesses.

With respect to the processes of the prior art, in particular with respect to WO01/12583, the reduction from ketone to chiral alcohol can be carried out without the use of hydrogen, with the resulting reduction of the risk, in particular at the industrial level and the possibility of making use of conventional equipment, without the need of special reactors which are necessary when working with hydrogen under pressure instead, such as for example in WO01/12583.

With respect to the yield and purity, the molar yield of the reduction set forth in WO01/12583 is 75%, whereas the yield of the reduction with the process of the invention reaches up to 90%, a difference that for an industrial production is highly significant, in particular because at the same time it allows to obtain the compounds of formula (I) with extremely high enantiomeric excesses and purities higher than 99%, fact that is fundamental considering that many compounds of formula (I) are used in the pharmaceutical field.

All these advantages make the process of the invention and the novel intermediate compounds a real and significant technical advancement with respect to the actual knowledge.

The following Experimental Section describes in details the process of the invention, only by way of example and not limitedly.

The invention is described herein particularly with reference to the preparation of (R) isomers of the compounds of formula (1) and of the compounds of formula (V), (VI) and (VII), but it is clear to the person skilled in the art that by using the (S)-tetrahydro-1-methyl-3,3-diphenyl-1H,3H-pyrrole[1,2-c][1,3,2]oxazaborole instead of the (R)-tetrahydro-1-methyl-3,3-diphenyl-1H,3H-pyrrole[1,2-c][1,3,2]oxazaborole, the (S) isomers of said compounds are obtained.

Experimental Section

Abbreviations

UPLC Ultra Performance Liquid Chromatography

UPLC-MS Ultra Performance Liquid Chromatography-Mass

NMR Nuclear Magnetic Resonance

DMSO dimethylsulfoxide

THF tetrahydrofuran

CBS Corey-Bakshi-Shibata catalyst

DCM dichloromethane

DMS borane-dimethyl sulfide

IPA isopropyl alcohol

EtOAc ethyl acetate

Cbz carbobenzyloxy group (—C(═O)—O-benzyl)

Analytical Methods

UPLC-MS

UPLC-MS: Waters Acquity™ Ultra Performance LC

Method 1:

Stationary phase: Acquity UPLC™ BEH SHIELD RP18, 1.7 um 2.1×50 mm

Column;

Mobile phase: A: H₂O+0.05% TFA; B: ACN+0.05% TFA;

Gradient: 5-100% B in 3 min; 100% B, 1 min

Flow 0.5 mL/min

Method 2:

Stationary phase: Acquity UPLC™ HSS T3, 1.8 um 2.1×50 mm Column;

Mobile phase: A: H₂O+0.05% TFA; B: ACN+0.05% TFA;

Gradient: 0-45% B in 3.50 min; 45-100% B from 3.50 to 4 min.

Flow 0.5 mL/min;

NMR

AV 300 MHz Bruker

Solvent: DMSO-d6

Temperature: 298K

Chiral HPLC:

HPLC: Agilent 1260

Stationary phase: Chiralpak OD-H 250×4.6 5 um

Mobile phase: A: Heptane 85%; B: Ethanol 15%

Gradient: Isocratic

Flow: 1 mL/min;

Column Temperature 25° C.

Wavelength: 220 nm

### EXAMPLE 1

### Preparation of benzyl(R)-(2-(3,4-dihydroxyphenyl)-2-hydroxyethyl)(methyl)carbamate

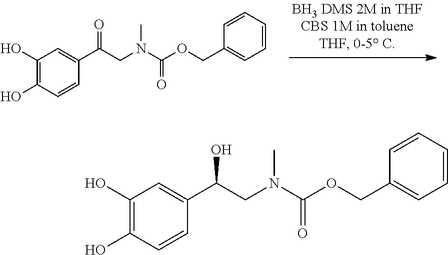 [SMILES: CN(CC(=O)c1ccc(O)c(O)c1)C(=O)OCc1ccccc1 ; CN(C[C@H](O)c1ccc(O)c(O)c1)C(=O)OCc1ccccc1]

A 2 M solution of borane-dimethyl sulfide in THF (1.5 mL, 1.24 eq) is added to a 1 M solution of (R)-tetrahydro-1-methyl-3,3-diphenyl-1H,3H-pyrrole[1,2-c][1,3,2]oxazaborole (3 mL, 1.24 eq) in toluene. A 0.15 M solution of benzyl (2-(3,4-dihydroxyphenyl)-2-oxoethyl)(methyl)carbamate (760 mg, 1 eq) in THF (16 mL) is slowly added by keeping the temperature below 2° C. and it is stirred until the disappearance of the reagent. 2 N HCl (aq) is added, toluene and water are added and the aqueous phase is separated. The organic phase is washed with 2 N HCl (aq), then with a NaHCO3 saturated solution and finally with a NaCl saturated solution, then it is dried over sodium sulfate. The solution is concentrated until obtaining a solid product which is filtered, obtaining 470 mg of benzyl (R)-(2-(3,4-dihydroxyphenyl)-2-hydroxyethyl)(methyl)carbamate as a white solid. Yield: 61%, purity (UPLC, UV 220 nm, method 1): 99%, chiral optical purity higher than 98%.

For analytical purposes the product has been purified by flash chromatography.

Mass and NMR confirm the structure:

UPLC MS (method 1): rt=1.38 min, m/z=318.47 (MH+)

¹H NMR (300 MHz, DMSO-d₆): δ ppm 8.77 (s, 2H), 7.29-7.41 (m, 5H), 6.74 (d, J=6.6 Hz, 1H), 6.62-6.70 (m, 1H), 6.45-6.59 (dd, J=7.8 Hz, J=18 Hz, 1H), 5.14-5.34 (br s, 1H), 5.00-5.10 (d, J=10.5 Hz, 2H), 4.51-4.62 (m, 1H), 3.22-3.31 (m, 2H), 2.78-2.87 (d, J=11.1 Hz, 3H).

### EXAMPLE 2

### Preparation of (R)-4-(1-hydroxy-2-(methylamino)ethyl)benzen-1,2-diol

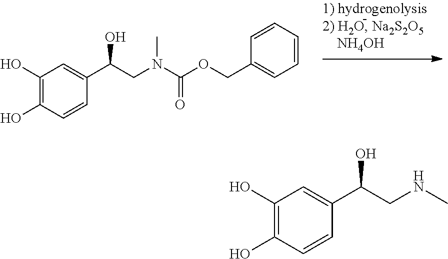 [SMILES: CN(C[C@H](O)c1ccc(O)c(O)c1)C(=O)OCc1ccccc1 ; CNC[C@H](O)c1ccc(O)c(O)c1]

Benzyl (R)-(2-(3,4-dihydroxyphenyl)-2-hydroxyethyl)(methyl)carbamate (430 mg, 1 eq) is solubilized in methanol (13 mL, 0.105 M), Pd/C 10% p/p (58 mg, 0.040 eq) and formic acid (160 uL, 3 eq) are added, and it is stirred at 50° C. for 1 hour. The reaction is left cooling at ambient temperature and the catalyst is filtered. The solution is concentrated and the residue retaken with an aqueous solution 2% p/p of sodium metabisulfite. Aqueous ammonia is added until an isoelectric pH and it is left under stirring for 1 h. The solid is filtered over Buchner, it is washed with water and dried under vacuum at 40° C. 185 mg of (R)-4-(1-hydroxy-2-methylamino)ethyl)benzen-1,2-diol are obtained as a white solid. Yield: 74%, purity (UPLC, UV 220 nm, method 2): 99.6%, optical purity higher than 98%.

Mass and NMR confirm the structure:

UPLC-MS (method 2): rt=0.80 min, m/z=184.15 (MH+)

¹H NMR (300 MHz, DMSO-d₆): δ ppm 6.72 (d, J=1.8 Hz, 1H), 6.64 (d, J=7.9 Hz, 1H), 6.55 (dd, J=8.1 Hz, J=1.7 Hz, 1H), 4.43 (dd, J=8 Hz, J=4.6 Hz, 1H), 2.43-2.58 (m, 2H), 2.29 (s, 3H).

### EXAMPLE 3

### Preparation of benzyl(2-(3,4-bis(benzyloxy)phenyl)-2-oxoethyl)(methyl)carbamate

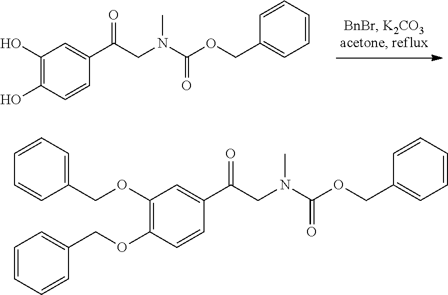 [SMILES: CN(CC(=O)c1ccc(O)c(O)c1)C(=O)OCc1ccccc1 ; CN(CC(=O)c1ccc(OCc2ccccc2)c(OCc2ccccc2)c1)C(=O)OCc1ccccc1]

To a suspension of 14.31 g of benzyl (2-(3,4-dihydroxyphenyl)-2-oxoethyl)(methyl)carbamate (CAS Registry Number: 101878-49-3) in acetone (0.29 M), K₂CO₃ (2.1 eq) and benzyl bromide (2.06 eq) are added. It is heated under reflux up to the disappearance of the starting product, the reaction mixture is filtered and the solvent evaporated. The resulting solid is crystallized in IPA/CH₃OH 3:1, after filtration and drying 19.8 g of benzyl (2-(3,4-bis(benzyloxy)phenyl)-2-oxoethyl)(methyl)carbarnate are obtained as a white solid.

Yield: 88%, purity (UPLC, UV 220 nm, method 1): 99.84%.

Mass and NMR confirm the structure:

UPLC MS (method 1): rt=2.48 min; m/z=496.13 (MH+)

¹H NMR (300 MHz, DMSO-d₆): δ ppm 7.56-7.68 (m, 2H), 7.15-7.51 (m, 16H), 5.27 (s, 2H), 5.21 (s, 2H), 5.01-5.11 (d, 2H), 4.75-4.80 (d, 2H), 2.84-2.98 (d, 3H).

### EXAMPLE 4

### EXAMPLE 4.1

### Preparation of benzyl(R)-(2-(3,4-bis(benzyloxy)phenyl)-2-hydroxyethyl)(methyl)carbamate

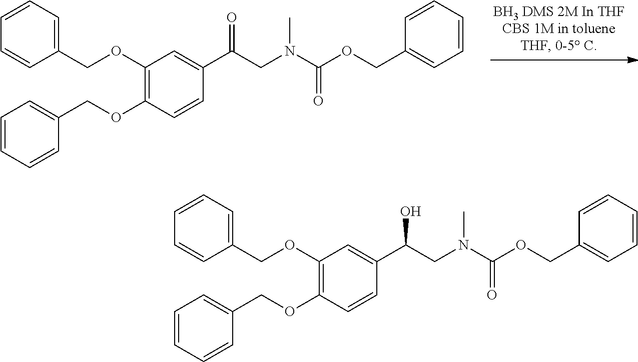 [SMILES: CN(CC(=O)c1ccc(OCc2ccccc2)c(OCc2ccccc2)c1)C(=O)OCc1ccccc1 ; CN(C[C@H](O)c1ccc(OCc2ccccc2)c(OCc2ccccc2)c1)C(=O)OCc1ccccc1]

A 2 M solution of borane-dimethyl sulfide in THF (20 mL, 1.28 eq) is added to a 0.79 M solution of (R)-tetrahydro-1-methyl-3,3-diphenyl-1H,3H-pyrrole[1,2-c][1,3,2]oxazaborole (CBS) (10.9 g, 1.25 eq) in toluene and cooled to about 0° C. A 0.3 M solution of benzyl (2-(3,4-bis(benzyloxy)phenyl)-2-oxoethyl)(methyl)carbamate (15.5 g, 1 eq) in THF is added and stirred until the completion of the reaction. Toluene is added and the reaction is quenched with 0.5 N HCl (aq). The organic phase is separated, which is washed and dried over Na₂SO₄. The solvent is evaporated under vacuum and 15.186 g of benzyl (R)-(2-(3,4-bis(benzyloxy)phenyl)-2-hydroxyethyl)(methyl)carbamate are obtained.

Yield: 97%, purity (UPLC, UV 220 nm, method 0:100%, Chiral purity 98% R enantiomer.

For analytical purposes, the product has been purified by flash chromatography over silica.

Mass and NMR confirm the structure:

UPLC MS (method1): rt=2.38 min; m/z=520.44 (M+Na)+; 480.39 (MH+−H2O)

¹H NMR (300 MHz, DMSO-d₆): δ ppm 7.25-7.50 (m, 15H), 6.93-7.11 (m, 2H), 6.72-6.87 (m, 1H), 5.31-5.46 (dd, J=4.3 Hz, J=16 Hz, 1H), 4.94-5.16 (m, 6H), 4.58-4.73 (m, 1H), 3.27-3.32 (m, 2H), 2.80 (s, 3H).

### EXAMPLE 4.2

### Preparation of benzyl(R)-(2-(3,4-bis(benzyloxy)phenyl)-2-hydroxyethyl)(methyl)carbamate

By operating as described in example 4.1 but using toluene instead of THF, the title compound is obtained with a chiral purity higher than 99%.

### EXAMPLE 5

### Preparation of (R)-1-(3,4-bis(benzyloxy)phenyl)-2-(methylamino)-ethan-1-ol hydrochloride

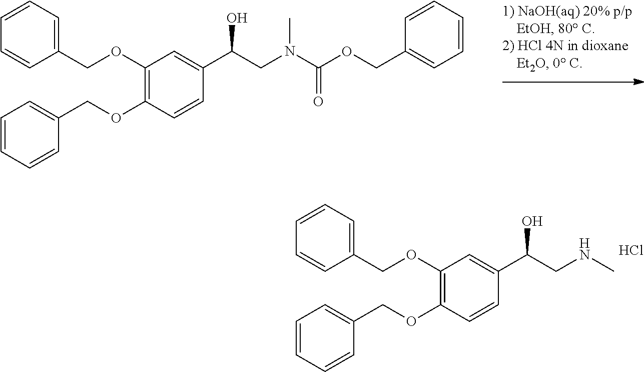 [SMILES: CN(C[C@H](O)c1ccc(OCc2ccccc2)c(OCc2ccccc2)c1)C(=O)OCc1ccccc1 ; CNC[C@H](O)c1ccc(OCc2ccccc2)c(OCc2ccccc2)c1 ; Cl]

A 20% solution of NaOH(aq) (21 mL, 19 eq) is added to a 0.1 M solution of benzyl (R)-(2-(3,4-bis(benzyloxy)phenyl)-2-hydroxyethyl)(methyl)carbamate (2.704 g) in EtOH and the mixture is stirred under reflux until complete conversion. It is diluted with toluene and water; the organic phase is washed and the solvent concentrated under vacuum. It is retaken with ethyl ether and 4 N HCl (1.77 eq) is added, obtaining the formation of a white precipitate. By filtering and drying under vacuum, 1.95 g of (R)-1-(3,4-bis(benzyloxy)phenyl)-2-(methylamino)-ethan-1-ol hydrochloride are obtained (white solid).

Molar yield: 90%, purity (UPLC, UV 220 nm, method 1): 99.59%.

Mass and NMR confirm the structure:

UPLC MS (method 1): rt=1.58 min; m/z=364.34 (MH+)

¹H NMR (300 MHz, DMSO-d₆): δ ppm 8.68 (s, 2H), 7.27-7.51 (m, 10H), 7.13 (d, J=1.7 Hz, 1H), 7.07 (d, J=8.2 Hz, 1H), 6.86-6.95 (m, 1H), 6.07 (d, J=3.9 Hz, 1H), 5.07-5.21 (m, 4H), 4.75-4.87 (m, 1H), 2.89-3.13 (m, 2H), 2.57 (s, 3H).

### EXAMPLE 6

### Preparation of (R)-4-(1-hydroxy-2-(methylamino)ethyl)benzen-1,2-diol

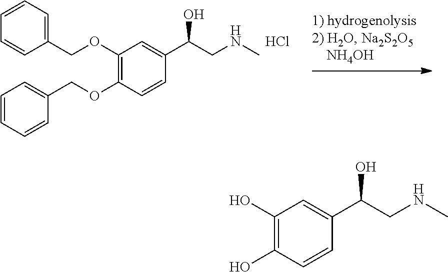 [SMILES: CNC[C@H](O)c1ccc(OCc2ccccc2)c(OCc2ccccc2)c1 ; Cl ; CNC[C@H](O)c1ccc(O)c(O)c1]

(R)-1-(3,4-bis(benzyloxy)phenyl)-2-(methylamino)-ethan-1-ol hydrochloride (2.095 g, 1 eq) is solubilized in methanol (50 mL, 0.105 M), Pd/C 10% p/p (200 mg, 0.039 eq) and ammonium formate (1.4 g, 4.6 eq) are added, and it is stirred in a closed system at 50° C. until the completion of the reaction. It is acidified with 4 N HCl and the solution is filtered. It is concentrated to a residue, which is retaken with water and aqueous ammonia is added until an isoelectric pH. The solid is filtered on Buchner, it is washed with water and dried under vacuum at 30° C. 830 mg of (R)-4-(1-hydroxy-2-methylamino)ethyl)benzen-1,2-diol are obtained as a white solid. Yield: 86%, purity (UPLC, UV 220 nm, method 2): 99.49%.

Mass and NMR confirm the structure:

UPLC MS (method 2): rt=0.82 min, m/z=184.21 (MH+)

¹H NMR (300 MHz, DMSO-d₆): δ ppm 6.72 (d, J=1.8 Hz, 1H), 6.64 (d, J=7.9 Hz, 1H), 6.55 (dd, J=8.1 Hz, J=1.7 Hz, 1H), 4.43 (dd, J=8 Hz, J=4,6 Hz, 1H), 2.43-2.58 (m, 2H), 2.29 (s, 3H).

### EXAMPLE 7

### Preparation of (R)-4-(1-hydroxy-2-(methylamino)ethyl)benzen-1,2-diol

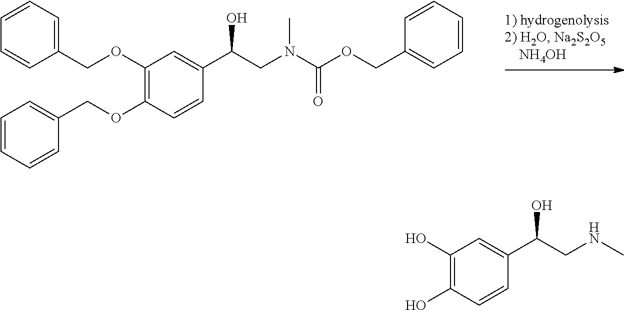 [SMILES: CN(C[C@H](O)c1ccc(OCc2ccccc2)c(OCc2ccccc2)c1)C(=O)OCc1ccccc1 ; CNC[C@H](O)c1ccc(O)c(O)c1]

Benzyl (R)-(2-(3,4-bis(benzyloxy)phenyl)-2-hydroxyethyl)(methyl)carbamate (4 g, 1 eq) is solubilized in methanol (90 mL, 0.09 M), Pd/C 10% p/p (330 mg, 0.039 eq) and formic acid (1.55 mL, 5 eq) are added, and it is stirred at 50° C. for 2 hours. The reaction is left cooling at ambient temperature and filtered over Celite. The solution is concentrated and the residue retaken with an aqueous solution 2% p/p of sodium metabisulfite. Aqueous ammonia is added until isoelectric pH. The solid is filtered on Buchner and dried under vacuum at 40° C. 1,2 g of (R)-4-(1-hydroxy-2-methylamino)ethyl)benzen-1,2-diol are obtained as a white solid. Yield: 81%, purity (UPLC, UV 220 nm, method 2): 99.93%. Optical purity higher than 99%

Mass and NMR confirm the structure:

UPLC MS (method 2): rt=0.89 min, m/z=184.21 (MH+)

¹H NMR (300 MHz, DMSO-d₆): δ ppm 6.72 (d, J=1.8 Hz, 1H), 6.64 (d, J=7.9 Hz, 1H), 6.55 (dd, J=8.1 Hz, J=1.7 Hz, 1H), 4.43 (dd, J=8 Hz, J=4.6 Hz, 1H), 2.43-2.58 (m, 2H), 2.29 (s, 3H).

### EXAMPLE 8

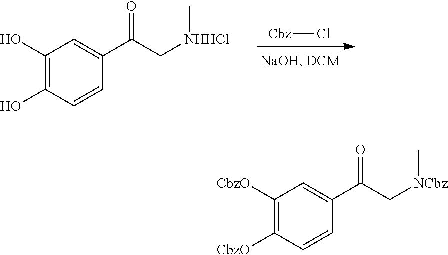

### Preparation of benzyl(2-(3,4-bis(((benzyloxy)carbonyl)oxy)phenyl)-2-oxoethyl)(methyl)carbamate

A suspension of adrenalone hydrochloride (2 g, 1 eq.) (CAS Registry Number: 62-13-5) in dichloromethane (4 ml) is cooled to 2° C. and 14.2 ml of 2 N NaOH are slowly added, by keeping T<7° C. By keeping the temperature between 5° C. and 0° C., a solution of Cbz-Cl in DCM (4.14 ml of Cbz-Cl, 3.1 eq. in 22.8 ml of DCM) and 2 N NaOH (17.5 ml) are simultaneously slowly dropped. At the end of the addition it is left for 2 h under vigorous stirring at a T of 5° C. The organic phase is separated, which is washed with water (2×25 ml) and a saturated solution of NaCl, dried over Na₂SO₄ and the solvent evaporated under vacuum. The crude product is purified by gravimetric chromatography over silica by eluting with Hexane/EtOAc (80/20 to 60/40 respectively), thus obtaining 4.3 g of benzyl (2-(3,4-bis(((benzyloxy)carbonyl)oxy)phenyl)-2-oxoethyl)(methyl)carbamate as a white solid. Yield: 80%, purity (UPLC, UV 220 nm, method 1): 96%.

For analytical purposes, the product has been purified by flash chromatography over silica.

Mass and NMR confirm the structure:

UPLC MS (method 1): rt=2.47 min; m/z=584.20 (MH+)

1H NMR (300 MHz, DMSO-d₆): δ ppm 8.04-8.09 (m, 1H), 7.94-8.03 (m, 1H) 7.42-7.65 (m, 1H), 7.21-7.37 (m, 15H), 5.28 (s, 4H), 5.02-5.12 (d, 2H), 4.83-4.89 (d, 2H), 2.91-2.96 (d, 3H).

### EXAMPLE 9

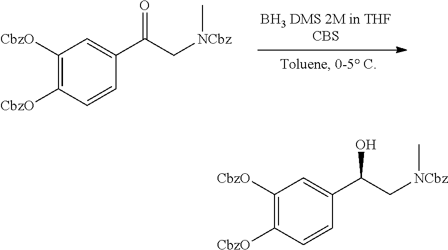

### Preparation of (R)-benzyl (2-(3,4-bis(((benzyloxy)carbonyl)oxy)phenyl)-2-hydroxyethyl)(methyl)carbamate

A 2 M solution of borane-dimethyl sulfide in THF (1.05 mL, 1.25 eq) is added to a solution of (R)-tetrahydro-1-methyl-3,3 -diphenyl-1H,3H-pyrrole[1,2-c][1,3,2]oxazaborole (CBS) (0.593 g, 1.25 eq) in 4.2 ml of toluene a cooled to about 0° C. A 0.25 M solution of benzyl (2-(3,4-bis(((benzyloxy)carbonyl)oxy)phenyl)-2-oxyethyl)(methyl)carbamate (1 g, 1 eq) in 7 ml of Toluene is added and stirred until completion of the reaction. Toluene (20 ml) is added and the reaction in quenched with 0.5 N HCl (aq). The organic phase is separated, which is washed with water and a saturated solution of NaCl, dried over Na₂SO₄ and the solvent evaporated under vacuum. The crude product is purified by gravimetric chromatography over silica by eluting with 80/20 toluene/EtOAc, thus obtaining 860 mg of (R)-benzyl-(2-(3,4-bis(((benzyloxy)carbonyl)oxy)phenyl)-2-hydroxyethyl)(methyl)carbamate as a straw yellow oil. Yield; 86%, purity (UPLC, UV 220 nm, method 1): 96% Chiral purity 98% R enantiomer.

For analytical purposes, the product has been purified by flash chromatography over silica.

Mass and NMR confirm the structure:

UPLC MS (method 1): rt=2.37 min; m/z=586.23 (MH+)

¹H NMR (300 MHz, DMSO-d₆): δ ppm 7.15-7.41 (m, 18H), 5.66-5.71 (dd, J=5.6 Hz, J=16 Hz, 1H), 5.25 (s, 4H), 4.97-5.06 (d, 2H), 4.83-4.78 (m, 1H), 3.32-3.36 (m, 2H), 2.85 (s, 3H).

### EXAMPLE 10

### Preparation of (R)-4-(1-hydroxy-2-(methylamino)ethyl)benzen-1,2-diol L-tartrate

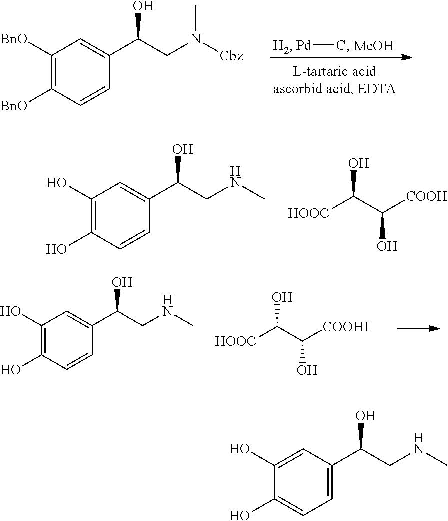

L-tartaric acid (8.3 g, 1.1 eq), ascorbic acid (100 mg) and acidic EDTA (50 mg) are charged into the inertized reactor. The solution in MeOH (500 mL) is added to benzyl (R)-(2-(3,4-bis(benzyloxy)phenyl)-2-hydroxyethyl)(methyl)pcarbamate of the example 4.2 (25.0 g, 1 eq) and the mixture is heated to 37° C. The (Pd-C 5%, 50% wet, 2.5 g 10% p/p) catalyst is charged and placed into a hydrogen atmosphere (absolute p=3.0±0.2 bar). It is left reacting until the complete consumption of hydrogen (ca. 3 L). The reactor is discharged by filtering the catalyst over cellulose and washing with MeOH (50 mL). The solvent is distilled under vacuum (T<50° C.) until a residue. The white solid is retaken with (IPA) (10 volumes over theoretical) and left under stirring at ambient temperature for 1 h, then cooled to 15-20° C. After 1.5 h it is filtered by washing with IPA (1 volume). The solid is dried in vacuum oven at 50° C. for 16 h. Yield: 93% (white solid).

The bitartrate salt (10.0 g) is redissolved in deionized H₂O (100 mL). Sodium metabisulfite is added and cooled to 5-10° C. The pH of the mixture is adjusted to 8.5 with aqueous ammonia. It is left under stirring for 30 minutes, then filtered and washed with deionized H₂O (10 mL) and MeOH (10 mL). Quantitative yield, e.e. >99.5%.

1. A process for the preparation of an optically active compound of Formula (I) 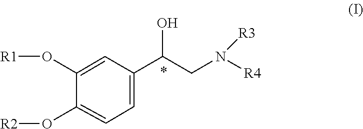
or a salt thereof, wherein
the asterisk means that the chiral carbon is in the optically active form (R) or (S);
R₁ e R₂ are, each independently, selected from hydrogen and a hydroxy protecting group; or R₁and R₂ together with the oxygen atoms to which they are bound, may form a protecting group in the form of a fused ring with benzene;
R₃ is selected from hydrogen and a protecting group of the amine function;
R₄ is selected from hydrogen and a C₁-C₄ alkyl;
said process comprising
a. reducing the compound of Formula (II) 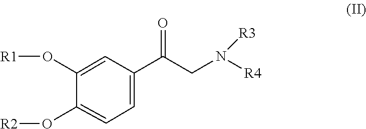
wherein R₁, R₂, R₃ e R₄ are as defined above; and when R₃ is hydrogen, the amine group may be salified,
said reduction being performed with a reducing complex made of boranes in presence of the Corey-Bakshi-Shibata (CBS) catalyst, in an organic solvent;
b. optionally, when R₁, R₂ and R₃ are protecting groups, removing said protecting groups to obtain the compound of formula (I) wherein R1, R2 and R3 are hydrogen and R4 hydrogen or a C1-C4 alkyl; and
c. optionally, converting the compound of formula (I) into a salt thereof;
steps (b) and (c) may be reversed.

2. The process according to claim 1, wherein said protecting groups may be removed by hydrogenation or alkaline hydrolysis.

3. The process according to claim 2, wherein said protecting groups are selected from benzyl and carbobenzyloxy.

4. The process according to claim 2, wherein said protecting groups are removed by hydrogenation with a maximum hydrogen pressure of 3.0±0.2 bar, in the presence of a carboxylic acid which has at least one chiral center and is in enantiomerically pure form.

5. The process according to claim 4 wherein said acid is selected from D-tartaric acid, L-tartaric acid, D-benzoyltartaric acid, L-benzoyltartaric acid, D-camphor-10-sulfonic acid, L-camphor-10-sulfonic acid, D-mandelic acid, L-mandelic acid.

6. The process according to claim 1, wherein R₁and R₂ are the same; and/or R₁ and R₂ do not both represent hydrogen; and/or R₁ and R₂ are the same and each represents a benzyl group.

7-8. (canceled)

9. The process according to claim 1, wherein R₃ represents a carbobenzyloxy group.

10. The process according to claim 1, wherein R₁ and R₂ are the same and each represents a benzyl group and R₃ represents a carbobenzyloxy group.

11. The process according to claim 1, wherein R₁, R₂ and R₃ are the same and each represents a carbobenzyloxy group.

12. The process according to claim 1, wherein R₃ is a carbobenzyloxy group and R₄ is hydrogen.

13. The process according to claim 1, wherein said alkyl group is selected from methyl and isopropyl.

14. The process according to claim 1, wherein R₁, R₂ and R₃ are the same and each represents a carbobenzyloxy group and R₄ is a methyl group.

15. The process according to claim 1, wherein R₃ is a carbobenzyloxy group and R₄ is a methyl group.

16. The process according to claim 1, wherein R₁ and R₂ each represents a benzyl group, R₃ is hydrogen or a carbobenzyloxy and R₄ is a methyl group.

17. The process according to claim 1, wherein R₃ is hydrogen and the compound of formula (II) is salified.

18. The process according to claim 1, wherein the reaction of step (a) is performed with a reducing complex made of CBS and borane (BH₃) and that said solvent is an apolar organic solvent, preferably selected from toluene and tetrahydrofuran.

19. The process according to claim 1, wherein said reducing complex is used in a substoichiometric amount.

20. The process according to claim 1, for the preparation of a compound of formula (I) wherein R_{1,} R₂ and R₃ each represents hydrogen and R₄ is a methyl group (epinephrine).

21. The process according to claim 20, wherein R₁ and R₂ are the same and each represents a benzyl group and R₃ represents a carbobenzyloxy group, wherein said protecting groups are removed by hydrogenation with a maximum hydrogen pressure of 3.0±0.2 bar and in presence of L-tartaric acid.

22. A compound selected from the compounds having the following formulas (III), (IV), (V), (VI) and (VII): 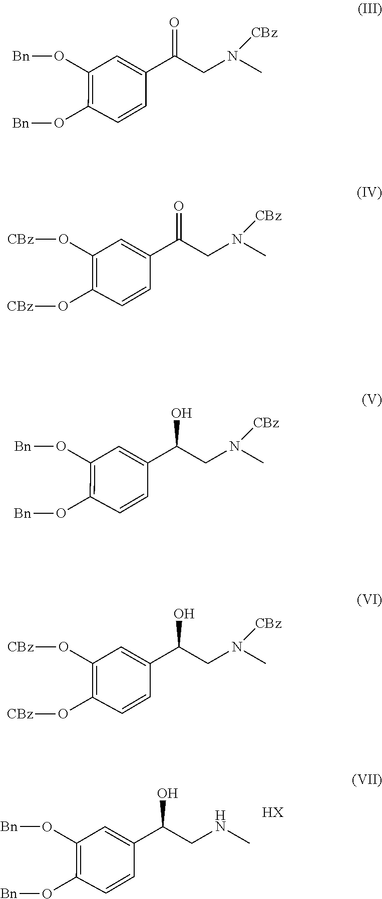
wherein X represents a halogen atom, advantageously bromine and chlorine, preferably chlorine, the compounds (V), (VI) and (VII) may be in the form of racemates, pure isomers or isomer mixtures, preferably in the form of (R) isomer.

23-24. (canceled)
